SEQUENCE LISTING (12 sequences)

SEQ ID NO: 1 - DNA, 3266 bp, Homo sapiens
ggcttccggttccgccacggcccagaggctgtgttgaggctaatggcggtgcccacggaactggatggagggagtgttaaggagaccgcagcggaagaggaatcgcgagttctggcacctggcgccgccccgttcggaaattttcctcattattctcgcttccaccctccggagcaacggctccgcctcctgcccccggagctgcttcgacagctctttcctgagagtcccgagaacgggccgattctggggctcgacgtggggtgtaactccggggatctgagtgtggctctatacaaacacttcctctccctacctgacggggaaacctgctcagatgcctcaagagaattccgtctcctctgctgcgacatagatccagtcctggtgaagcgagccgaaaaagaatgtccttttcctgatgccttgacttttatcaccctggacttcatgaatcaaaggacccggaaggttctcttgagctctttcttaagccaatttggacgttcagtttttgacattggcttctgcatgtcaataaccatgtggattcatctgaatcatggagaccatggcctatgggagttcctggcccatctttcctccctctgccactacctccttgtggagccccaaccctggaagtgttaccgggcagctgcaaggcgtctccgaaagctgggactccatgattttgaccacttccactcccttgccatccgaggtgacatgcccaatcagattgtgcagatcttgacccaggatcatggcatggaattaatatgttgctttggcaacaccagttgggacagaagccttctgctcttcagggcaaaacaaaccatagagactcatccaatccctgaatcactgatagaaaaagggaaagaaaagaacagattaagtttccagaagcagtgagatgggagggcaagaagaccaagaaagatattgaaagggttttatattgagaattatgttcattctccttaattccttaacaatcaggcagccgcaaaacctggcaggagcttttggtaaaatgtccaaggcacataattgaaagaatcagtatctgttccccattgtttggaacaaatcttgaggagaatgaatctgtggagtgatgagctaggatatctggattgagatggcctaggaaaagtggtcctggttatgggttatggagagggctgcttttggggattatttataaagatcagactaggctagtcttaaaatatgtcagttcctgggctctctgaatcctacctggtttcttcaggcttctggacttgctaggcctgcctttgggcaaaaccttagtaattcaggtctggaatggggcttaggaatctattttctgtttttttgtttgtttgtttttgcattcttctattgtaatatatacataaaatttaccattttaaccgtttttaagtacagtttttcagaacgtggaaatcccatcaaaaattggattaaaaaattaaaagataaattgataatttttttaaaagtatacaattttgtgatattaagtacatttcacaatgaggcatctatttttaacagctcccccagatgattctgaggcaagcagaggtagaccatactttgagtgcttacggttccattcagtccttctctatgtatttttcctggggtgagttcttccctatggttttaacaactacctacatgcggccgggcgcggtggctcacgcctgtaatcgcagcactttgggaggccgaggcgggtggatcacgaggtcaggagatcgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaacaaaaaaaaaaacaactacctacatgcttatggtccccaaatatattggtagcccgacatccacagttccagactagcatttttcctgttttgtcattatatcccttagttcttaggtacatcagaatcaacagatctcaaaagcaagctcactaccctcattcaaaacctagtcctatctcatttgatagtaatcattgcttatgctagaaacctcagttgattttctcacttacactcccccacattgaaatgctctcaaagtccagcagcaaattctatcaattacatgtaagtttcattccatacatgtgtccggtgcctggtgggaggttcctatattaaagtcatctaccttccatccattgtctgccttacagccacagtttactcatctgtacaacgaaataacaccactttccttttcctatgcccaaatgagctaaggatgtgaaggtattttgcagaatatagagtgataaagaaaaataaggtgctattacactgtgtgagcatgccgcagaccacaactgcacttgttagactggaggcttatgagaaaaacatctcaccttgtagaaaacatgcatcttactatctctgataacttgtttttcaggatgatgaagtaataaggagtccagcttcagtgctacagagcattttaaaggaagcagtgttgggagggagggatatggaactaatggaaatgaggctagtttatgatggtctcagttatcacagtagatacagagctgagtttacccacaagatcttaatttaggaacactttgaaaccctgtaaccctgctgggtgctctctcctctcactccaccaaggcagcgcacagaaacatacatggggctgtggtaggatgaataatggtcccccaagatgtccacctcctttagcaaaaggggctttgcaaatgagattgaggatcttcagatagggagattaatctggatgatcagaatataccctaagtgcaatcatcagtgttcttataatagggaggcagagggagagctgaccacagtaggagatgtaatgacaaaagcaagaggttggagtgatatgaagaagggctgcaagtcaaggaatgcaggtggcttctagaagttagaaaaagcaagaaaatgtattctcccctgaagcctccagaaggaacagccctgccaacaccttgatttcagatttaatctccagagctgtaagagaataaatcagtattttaaaccaaaaaaaaaaaaaaaaaa

SEQ ID NO: 2 - protein, 292 aa, Homo sapiens
MAVPTELDGGSVKETAAEEESRVLAPGAAPFGNFPHYSRFHPPEQRLRLLPPELLRQLFPESPENGPILGLDVGCNSGDLSVALYKHFLSLPDGETCSDASREFRLLCCDIDPVLVKRAEKECPFPDALTFITLDFMNQRTRKVLLSSFLSQFGRSVFDIGFCMSITMWIHLNHGDHGLWEFLAHLSSLCHYLLVEPQPWKCYRAAARRLRKLGLHDFDHFHSLAIRGDMPNQIVQILTQDHGMELICCFGNTSWDRSLLLFRAKQTIETHPIPESLIEKGKEKNRLSFQKQ

SEQ ID NO: 3 - DNA, 4107 bp, Homo sapiens
gcgcaaccctccggaagctgccgcccctttccccttttatgggaatactttttttaaaaaaaaagagttcgctggcgccaccccgtaggactggccgccctaaaaccgtgataaaggagctgctcgccacttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaatacagggaaaaaaaaaaaaaaaaaa

SEQ ID NO: 4 - protein, 350 aa, Homo sapiens
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGIRNVLLAFAGVAGVGAGLAYLIR

SEQ ID NO: 5 - DNA, 2379 bp, Homo sapiens
gacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaaaaaaaaaaaaaaa

SEQ ID NO: 6 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 7 - DNA, 4785 bp, Homo sapiens
acctgcttccctctcggatagccagggctgatgatttcaactcattaatcattccgaactcttcccaacaaacagcctcatggctgtcccggacctcctctgtcccggctcaggaaatgatctcggtcggaagtgaaaagaccaaatcggccccaggaggccattttcttcgacagaagctgaggcgtgggggcgtggccttcccccgcttccccctccactcccccgcccgcccccggccccggctttccgatctcggcccagggaagagccttggtcgcgaatcaccagtttgcatttgcccctggggcgggggcggctttctgagaccaggctggggctgggactccgccaccttcagctaagggtaccgaggtcagtccgggagtcgcgtcaccgggactcgaacccgcctcttcgtcttccgaggcctagtgcgagcctgcggcgctaaacgcagtcacactgtccctttaaggcggcactttttattcttcatttgtttacttcttcatgcgctcttcggtctttacaaaatgtgctcgcgatatccttccgcaagtgtcagtctcaggcactcaccacccgccttgaagccatttctataatcttaagactcgcgagttagcagccgtgggccgacgtctctctttataggatttaattcttttacattttagggactactttttaggaataaaccattcttgcgttagttaataaaggttcataaccttcggaacccctccgctgagaacaaaatacgataatagcctttataacgcaccttttcataaaaatattctgtgaggactctcataggccatcgaatcttctcgtattcctgaaacagaatggtacggtccagaacccgccccatcttctctgcgattggccgtccttcctcagcgagtcgtgtgatttttcaccaatggccgctcgcgtttctttagcgaggtctaagcgatggaaggtggcggccagggagacgccccctacgcgtcctgggattggctacgtcgcacggcgcgcgagggcggcgggcccgaaagataagaactacgactcccggcgccccgcaaagggaactccgttacgcatgcgcacgggcggggcgaaaaagcttctatataaaaggggcgcagaggactgggagacagcagttggaagttggcaggtggagaggcaggttgggagggaaagtcgggggaggacgcggaagaggagctgtgggaagggggaggagggagggaggaaaagaggaggaggcggaggagaactgagcagagcagagcatcgagccaaaggggagatgagtttgtctgtcctctgctgaggctacggccgggcctagggaactgggagcttgggtggaagcgacacccgtggaagtgggaggaggtggcgccgggactttaaccccttgtgggctctgcggcaggggatttaaccctttgtggatctggcccctcggaggcagcgtcatcggtagttttaaccccttcggggctgggtttcacgcactggacttaccctcatcaccttgctcaccaactcctttattggggtgctccgcttggaggtttgaggcccacctccgcccattacgtactgttcctgccgctgcacccccttggacccgctagctggccgcactgtgggcgcttaaccctttactgacttgagctccccagattgcagttggagtttgctgatagaaggactagctaaaggcgtcactgcaggaattacaaactgaagaggactctgttggactgttttttttttctttttcttttttttaagaaaaacccatttttttccttaaggacttactagccaaaatttcttaaacttcgaggactctactagccatggccgagccattcttgtcagaatatcaacaccagcctcaaactagcaactgtacaggtgctgctgctgtccaggaagagctgaaccctgagcgccccccaggcgcggaggagcgggtgcccgaggaggacagtaggtggcaatcgagagcgttcccccagttgggtggccgtccggggccggagggggaagggagcctggaatcccaaccacctcccttgcagacccaggcctgtccagaatctagctgcctgagagagggcgagaagggccagaatggggacgactcgtccgctggcggcgacttcccgccgccggcagaagtggaaccgacgcccgaggccgagctgctcgcccagccttgtcatgactccgaggccagtaagttgggggctcctgccgcagggggcgaagaggagtggggacagcagcagagacagctggggaagaaaaaacataggagacgcccgtccaagaagaagcggcattggaaaccgtactacaagctgacctgggaagagaagaaaaagttcgacgagaaacagagccttcgagcttcaaggatccgagccgagatgttcgccaagggccagccggtcgcgccctataacaccacgcagttcctcatggatgatcacgaccaggaggagccggatctcaaaaccggcctgtactccaagcgggccgccgccaaatccgacgacaccagcgatgacgacttcatggaagaagggggtgaggaggatgggggcagcgatgggatgggaggggacggcagcgagtttctgcagcgggacttctcggagacgtacgagcggtaccacacggagagcctgcagaacatgagcaagcaggagctcatcaaggagtacctggaactggagaagtgcctctcgcgcatggaggacgagaacaaccggctgcggctggagagcaagcggctgggtggcgacgacgcgcgtgtgcgggagctggagctggagctggaccggctgcgcgccgagaacctccagctgctgaccgagaacgaactgcaccggcagcaggagcgagcgccgctttccaagtttggagactagactgaaacttttttgggggagggggcaaaggggactttttacagtgatggaatgtaacattatatacatgtgtatataagacagtggacctttttatgacacataatcagaagagaaatccccctggctttggtttcgtaaatttagctatatgtagcttgcgtgctttctcctgttcttttaattatgtgaaactgaagagttgcttttcttgttttcctttttagaagtttttttccttaatgtgaaagtaatttgaccaagttataatgcatttttgtttttaacaaatcccctccttaaacggagctataaggtggccaaatctgagaacaattaaattcattttagttataataaatttaatatttgtaaatgtaacatagtttcagtgtgatttctagagctaattcaaaatagtattgatatattttatgtgactgcatttttggggaggggtaccgaaatcgttaaatttgtcagtttgcaaaaatatcaatctttaatgggagaattttcaatttgccaattttttccttgaatgggtttaagtatgctacaatatacagttcaggcaaaatttaagatgtaattatcttcaatacttaagtgtgcttgctttctagtgccttggttttctttcttgatgctggaaaaataaacaaaccggtattgagtgtttaggcgagtggaaagtggctacaatccaaaattttaaatttaactctgcctcggccattcaaaagtctaataacaaaaaatgtaaacctaatttggcagtttgttaggttagacaactgacagcctcatttcattcctacaagttggttttcagtaatctcttccttccccccagtaaggctggaagaggctcttggcaaacttcttagtgcaagcaatggttagattaatttgtgaggcagctctttaagacgttcagaggtaagaaatactggatttataaagcaaatggctgtttgggggattccaaggatttacctaattgtccaattctacgtgctctctataccaaaacaaaaaaaaaaagctatccacctttccatgtgggtcaaactaaaattagaaatgtcccctcactgcagatcaaatgtaaagcttccagttaaggagctaaatgaggtcctcagctgaatgaggaaccctgtacatccccttgcacagccctattctaaatcgcttaaactatgctgatagctgcttaggttcttgagtagttctgctcttaaacgtagggaggccctgagaactaaattttgccccaaaataaaaacagaaattatgagattgcctcctgtcattttggttaacccagtccttcacctgccctgtgtcagtgtcttctgagggcaattgcgttgctcaaatcactagcacagaggttccttaatttggggccttagaaaccattgtgggccttggggtccatgaaccccatgaaattatttgtagacttgtatgtacatttttctggggagaaggttcaagagattcataagattgtcaaactccttgaaggttcagaacctctgcagggaagggggaagaaaaccctcccattaggaagcatgcttttgcagttaaatggcgatggtggaggtgatagggacttcaagagtaaaatgcaccttgtattgcataagaagcatacacaaatcaataaatcaagggagattataccagtaggactgaatcagggccttcaaagctggactgagttggtcctgttctggcacatatggtccactggagacaatgtatgattgagtttttctttggtctaaaaattatattaaacatttattttgaaata

SEQ ID NO: 8 - protein, 359 aa, Homo sapiens
MAEPFLSEYQHQPQTSNCTGAAAVQEELNPERPPGAEERVPEEDSRWQSRAFPQLGGRPGPEGEGSLESQPPPLQTQACPESSCLREGEKGQNGDDSSAGGDFPPPAEVEPTPEAELLAQPCHDSEASKLGAPAAGGEEEWGQQQRQLGKKKHRRRPSKKKRHWKPYYKLTWEEKKKFDEKQSLRASRIRAEMFAKGQPVAPYNTTQFLMDDHDQEEPDLKTGLYSKRAAAKSDDTSDDDFMEEGGEEDGGSDGMGGDGSEFLQRDFSETYERYHTESLQNMSKQELIKEYLELEKCLSRMEDENNRLRLESKRLGGDDARVRELELELDRLRAENLQLLTENELHRQQERAPLSKFGD

SEQ ID NO: 9 - DNA, 2303 bp, Homo sapiens
aagccaaaaaccgccgctttcaaatcgccaatcaaattcttccttggcttcgagtctctcagccggccgcgctctccgatgcccagccctcctggaaccacctcgcctgtgacgtaggtggagcgcgcactgcctccgggcccgtctttctcaattgggaccggaaaacgttgtcgctcatcctatgacgcgaaagtaaccgagactatcaggatccggagacggaaatgtccgaaggccgcagtacttgaccctgtattttgggagtcgaacggagaatggaaactgaaagtggaaatcaggaaaaggtaatggaagaagaaagcactgaaaagaaaaaagaagttgaaaaaaagaaacggtcacgagttaaacaggtgcttgcagatattgctaagcaagtggacttctggtttggggatgcaaatcttcacaaggatagatttcttcgagaacagatagaaaaatctagagatggatatgttgatatatcactacttgtgtcttttaacaaaatgaaaaaattgactactgatgggaagttaattgccagagcattgagaagttcagctgttgtagagcttgatttggaaggcaccagaatccggaggaaaaaacctctgggggaaagaccaaaggatgaggatgaacgcacagtgtatgtggagttacttcccaaaaatgttaatcacagctggattgaaagagtatttgggaaatgtggcaatgttgtttatataagtataccacattataagtctactggagatccaaagggatttgcgtttgtggaatttgaaacaaaagaacaagcagcaaaagcaattgagtttcttaacaacccaccagaagaagcaccaagaaaacctggcatatttcctaaaacagtgaaaaataagcccattccagccttaagagttgtggaagagaagaaaaagaaaaagaagaagaaaggccgaatgaaaaaggaagacaatatccaagccaaagaagaaaacatggacacaagcaacaccagcatcagtaaaatgaaaagatccagacccacatctgagggctctgacattgagtccactgaaccccaaaagcagtgctcaaagaaaaagaaaaaacgggacagagttgaagcatctagcttacctgaagtcagaacagggaagaggaagagaagcagctctgaagatgcagaatccctagctccccgatcaaaagtaaagaaaattattcagaaagacatcattaaggaagcatcagaagcttccaaggaaaatagagatatagaaatctctactgaagaggaaaaggatactggagatctaaaagatagctctctcttgaaaacaaaaaggaaacataagaaaaaacataaagagagacataaaatgggagaagaagttataccattaagagtgctatcaaagagcgaatggatggatttgaaaaaagagtatttagcgctacaaaaagctagcatggcttctttaaaaaaaacaatatcccaaataaaatcagagtcagaaatggaaacagacagtggagtacctcaaaacactggaatgaaaaatgaaaaaacagccaacagggaagagtgtcgcacccaggagaaagttaatgcaacaggaccacagttcgtgagtggagtgattgtgaagatcattagcacagagcctctacctggcaggaaacaagtccgggatactttggcagcaatctcagaagttctttatgttgatttgctagaaggggatacagaatgccatgctagatttaaaactcctgaggatgctcaagcagtaataaatgcctatacagaaattaacaagaaacactgctggaaactcgagatcctttctggtgatcacgaacaaaggtattggcagaagattttggttgatagacaggcaaaacttaatcagcctcgggaaaagaaaagaggcactgaaaagttaatcaccaaagctgaaaagattagactggcaaagactcaacaagcgagtaaacatataagattttctgaatatgattgaaaaaaaaaacagttcacctcttaatacttcacaagatacttgagctgttcttgggagattcacttttattatggtagcactgcataattaatgtgtttttaattaaaagaaatatctttgttcctcaacttgtaaataagacttttttctagagacaaatatgatgtataccacaatttttcttaaacattttatttgttgaaattatcttagatgtcagtgtcaggtgatttagtaaataaatgtgttttgaacattaaaaaaaaaaaaaaaaaa

SEQ ID NO: 10 - protein, 582 aa, Homo sapiens
METESGNQEKVMEEESTEKKKEVEKKKRSRVKQVLADIAKQVDFWFGDANLHKDRFLREQIEKSRDGYVDISLLVSFNKMKKLTTDGKLIARALRSSAVVELDLEGTRIRRKKPLGERPKDEDERTVYVELLPKNVNHSWIERVFGKCGNVVYISIPHYKSTGDPKGFAFVEFETKEQAAKAIEFLNNPPEEAPRKPGIFPKTVKNKPIPALRVVEEKKKKKKKKGRMKKEDNIQAKEENMDTSNTSISKMKRSRPTSEGSDIESTEPQKQCSKKKKKRDRVEASSLPEVRTGKRKRSSSEDAESLAPRSKVKKIIQKDIIKEASEASKENRDIEISTEEEKDTGDLKDSSLLKTKRKHKKKHKERHKMGEEVIPLRVLSKSEWMDLKKEYLALQKASMASLKKTISQIKSESEMETDSGVPQNTGMKNEKTANREECRTQEKVNATGPQFVSGVIVKIISTEPLPGRKQVRDTLAAISEVLYVDLLEGDTECHARFKTPEDAQAVINAYTEINKKHCWKLEILSGDHEQRYWQKILVDRQAKLNQPREKKRGTEKLITKAEKIRLAKTQQASKHIRFSEYD

SEQ ID NO: 11 - DNA, 2466 bp, Homo sapiens
atgttttacggaagccgatagtccttgctcagcggcaccccgtccttccggctctcggctttgccacaaagcttcccgaagacgcggccgctacccggagacgcggtcgccacccagaagcgctctcccgggaagccccgctcgtgggaccgcgccacctgcgccgcctctgcggcccgcagcccgacgggcgccgccatgttggggtcctagcgagggacgcgtaggtgtcttcataagatgccggggcagcggcgcgcgctttcccccaagatggcgtccatgcgggagagcgacacgggcctgtggctgcacaacaagctgggggccacggacgagctgtgggcgccgcccagcatcgcgtccctgctcacggccgcggtcatcgacaacatccgtctctgcttccatggcctctcgtcggcagtgaagctcaagttgctactcgggacgctgcacctcccgcgccgcacggtggacgagatgaagggcgccctaatggagatcatccagctcgccagcctcgactcggacccctgggtgctcatggtcgccgacatcttgaagtcctttccggacacaggctcgcttaacctggagctggaggagcagaatcccaacgttcaggatattttgggagaacttagagaaaaggtgggtgagtgtgaagcgtctgccatgctgccactggagtgccagtacttgaacaaaaacgccctgacgaccctcgcgggacccctcactcccccggtgaagcattttcagttaaagcggaaacccaagagcgccacgctgcgggcggagctgctgcagaagtccacggagaccgcccagcagttgaagcggagcgccggggtgcccttccacgccaagggccgggggctgctgcggaagatggacaccaccaccccactcaaaggcatcccgaagcaggcgcccttcagaagccccacggcgcccagcgtcttcagccccacagggaaccggacccccatcccgccttccaggacgctgctgcggaaggaacgaggtgtgaagctgctggacatctctgagctggatatggttggcgctggccgagaggcgaagcggagaaggaagactctcgatgcggaggtggtggagaagccggccaaggaggaaacggtggtggagaacgccaccccggactacgcagccggcctggtgtccacgcagaaacttgggtccctgaacaatgagcctgcgctgccctccacgagctaccttccctccacgcccagcgtggttcccgcctcctcctacatccccagctccgagacgcccccagccccatcttcccgggaagccagccgcccaccagaggagcccagcgccccgagccccacgttgccagcgcagttcaagcagcgggcgcccatgtacaacagcggcctgagccctgccacacccacgcctgcggcgcccacctcgcctctgacacccaccacacctccggctgtcgcccctaccactcagacacccccggttgccatggtggccccgcagacccaggcccctgctcagcagcagcctaagaagaacctgtccctcacgagagagcagatgttcgctgcccaggagatgttcaagacggccaacaaagtcacgcggcccgagaaggccctcatcctgggcttcatggccggctcccgagagaacccgtgccaggagcagggggacgtgatccagatcaagctgagcgagcacacggaggacctgcccaaggcggacggccagggtagcacaaccatgctggtggacacagtgtttgagatgaactatgccacgggccagtggacgcgcttcaagaagtacaagcccatgaccaatgtgtcctagaaccacctgcctcacagctggccgtcacttgtgggggtccacgggacgatggctttgccagcttaaagtaaccggatggcggacacctggcccccgaggtcccccggccgccgccctgctgctgacccagcctgttttaagttctggatgcatttctctggggtatttggggcttatttttaaaattttaatatgggttcttttttgtgtgatttaagacactttttggactcaacgttacatttttgaatgtagtaagtaaattaaccaaaaaagttacaacttcctaattttagtgacagctctgcctgttagactcttactttttaaaatcttttctattttccctcgctggggcagtgccctcctacccccagggttgaggggaccaaggtggcacggtggtactgggggtgcggcagggacacccgaccacaccagagcgtgggagacggtgggccttgtcccctgcctgtgcctgcctgggagttttgtattcatcttttgtatagttgtggacatttaagacagtctttgggtacctattttcattgtaaaactatctgaaccattaaagtcgagcttttctaaagaaagccaccaaaa

SEQ ID NO: 12 - protein, 528 aa, Homo sapiens
MASMRESDTGLWLHNKLGATDELWAPPSIASLLTAAVIDNIRLCFHGLSSAVKLKLLLGTLHLPRRTVDEMKGALMEIIQLASLDSDPWVLMVADILKSFPDTGSLNLELEEQNPNVQDILGELREKVGECEASAMLPLECQYLNKNALTTLAGPLTPPVKHFQLKRKPKSATLRAELLQKSTETAQQLKRSAGVPFHAKGRGLLRKMDTTTPLKGIPKQAPFRSPTAPSVFSPTGNRTPIPPSRTLLRKERGVKLLDISELDMVGAGREAKRRRKTLDAEVVEKPAKEETVVENATPDYAAGLVSTQKLGSLNNEPALPSTSYLPSTPSVVPASSYIPSSETPPAPSSREASRPPEEPSAPSPTLPAQFKQRAPMYNSGLSPATPTPAAPTSPLTPTTPPAVAPTTQTPPVAMVAPQTQAPAQQQPKKNLSLTREQMFAAQEMFKTANKVTRPEKALILGFMAGSRENPCQEQGDVIQIKLSEHTEDLPKADGQGSTTMLVDTVFEMNYATGQWTRFKKYKPMTNVS

(19) United States
(12) Patent Application Publication
(10) Pub. No.: US 2014/0005070 A1
(43) Pub. Date: Jan. 2, 2014

(54) MARKERS ASSOCIATED WITH CYCLIN-DEPENDENT KINASE INHIBITORS

(71) Applicants: Michel Faure, Oakland, CA (US); Zhenhai Gao, Orinda, CA (US); James Sutton, Pleasanton, CA (US); Guoying Karen Yu, Kensington, CA (US)

(72) Inventors: Michel Faure, Oakland, CA (US); Zhenhai Gao, Orinda, CA (US); James Sutton, Pleasanton, CA (US); Guoying Karen Yu, Kensington, CA (US)

(73) Assignee: Novartis AG, Basel (CH)

(21) Appl. No.: 14/004,623

(22) PCT Filed: Mar. 28, 2012
(86) PCT No.: PCT/IB2012/051482

§ 371 Date: Sep. 11, 2013

Related U.S. Application Data
(60) Provisional application No. 61/468,452, filed on Mar. 28, 2011.

(51) Int. Cl. C12Q 1/68

(52) U.S. Cl. CPC C12Q 1/6881
USPC 506/9

(57) ABSTRACT

The invention provides methods of monitoring differential gene expression of pharmacodynamic (PD) markers in a patient treated with a Cyclin Dependent Kinase Inhibitor (CDKI), methods of determining the sensitivity of a cell to a CDKI by measuring PD markers and methods of screening for candidate CKDI.

### FIELD OF THE INVENTION

The present invention relates to the field of pharmacogenomics, and the use of pharmacodynamic markers useful in following patient response to treatment, cancer sensitivity and screening of compounds.

### BACKGROUND

Protein kinases constitute a large family of structurally related enzymes that are responsible for the control of a variety of signal transduction processes within the cell. (Hardie, G. and Hanks, S. The Protein Kinase Facts Book, I and II, Academic Press, San Diego, Calif.: 1995). The kinases may be categorized into families by the substrates they phosphorylate (e.g., protein-tyrosine, protein-serine/threonine, lipids, etc.).

Many diseases are associated with abnormal cellular responses triggered by the protein kinase-mediated events described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, viral diseases, and hormone-related diseases. For example, Gleevec, a tyrosine kinase inhibitor has been successfully used in the treatment of chronic myelogenous leukemia and gastrointestinal stromal tumors.

The cyclin-dependent kinase (CDK) complexes are a class of kinases that are targets of interest. The CDKs participate in cell cycle progression and cellular transcription, and loss of growth control is linked to abnormal cell proliferation in disease (Malumbres and Barbacid, Nat. Rev. Cancer 2001, 1:222). Increased activity or temporally abnormal activation of cyclin-dependent kinases has been shown to result in the development of human tumors (Sherr C. J., Science 1996, 274: 1672-1677). Indeed, human tumor development is commonly associated with alterations in either the CDK proteins themselves or their regulators (Cordon-Cardo C., Am. J. Pathol. 1995; 147(3): 545-560; Karp J. E. and Broder S., Nat. Med. 1995; 1: 309-320; Hall M., Adv. Cancer Res. 1996; 68: 67-108).

CDK7 and CDK9 play key roles in transcription initiation and elongation, respectively (see, e.g., Peterlin and Price 2006, Cell 23: 297-305, Shapiro. J., Clin. Oncol. 2006; 24: 1770-83). Inhibition of CDK9 has been linked to direct induction of apoptosis in tumor cells of hematopoetic lineages through down-regulation of transcription of anti-apoptotic proteins such as MCL1 (Chao S.-H., J. Biol. Chem. 2000;275: 28345-28348; Chao, S.-H., J. Biol. Chem. 2001;276:31793-31799; Lam, Genome Biology 2001 2: 1-11; Chen, Blood 2005; 106:2513; MacCallum, Cancer Res. 2005; 65:5399; and Alvi, Blood 2005; 105:4484). In solid tumor cells, transcriptional inhibition by downregulation of CDK9 activity synergizes with inhibition of cell cycle CDKs, for example CDK1 and 2, to induce apoptosis (Cai, D.-P., Cancer Res. 2006, 66:9270). Inhibition of transcription through CDK9 or CDK7 has a selective non-proliferative effect on the tumor cell types that are dependent on the transcription of mRNAs with short half lives, for example Cyclin D1 in Mantle Cell Lymphoma. Some transcription factors such as Myc and NF-ₖB selectively recruit CDK9 to their promoters, and tumors dependent on activation of these signaling pathways may be sensitive to CDK9 inhibition.

Certain CDK inhibitors are useful as chemoprotective agents through their ability to inhibit cell cycle progression of normal untransformed cells (Chen, J., Natl. Cancer Instit., 2000; 92: 1999-2008). Pre-treatment of a cancer patient with a CDK inhibitor prior to the use of cytotoxic agents can reduce the side effects commonly associated with chemotherapy. Normal proliferating tissues are protected from the cytotoxic effects by the action of the selective CDK inhibitor.

Finding pharmacodynamic (PD) markers which indicate that a therapeutic is active can be valuable. Such markers can be used to monitor the response of those patients receiving the therapeutic. If a PD marker indicates that the patient is not responding appropriately to the treatment, then the dosage administered can be increased, decreased or completely discontinued. As such, there is a need to develop PD markers associated with CDK inhibitors. This approach ensures that patients receive the most appropriate treatment.

In the development of CKD inhibitors, a PD marker will aid in understanding the mechanism of action upon administration. The mechanism of action may involve a complex cascade of regulatory mechanisms in the cell cycle and this analysis is done at the pre-clinical stage of drug development in order to determine the particular activity of the candidate CDK inhibitor. Of particular interest in the pharmacodynamic investigation is the identification of specific markers of activity, such as the ones disclosed herein.

### SUMMARY OF THE INVENTION

The invention relates to the analysis that a number of genes identified in Table 2 act as specific pharmacodynamic (PD) markers for the activity of CDK inhibitors (henceforth “CDKI”). In particular the invention relates to the up or down regulation of the expression of the identified genes after CDKI treatment. PD markers are useful in determining that patients receive the correct course of treatment. The invention is an example of “personalized medicine” wherein patients are treated based on a functional genomic signature that is specific to them.

The invention comprises a method of monitoring response of a patient to treatment. The method includes the step of administration of any CDKI to the patient and measurement of gene expression of a biological sample obtained from the patient. The response of the patient is evaluated based the detection of gene expression of at least one biomarker from the Table 2. Detection and/or alteration in the level of expression of at least one PD marker compared to baseline may be indicative of the response of the patient to the treatment. The pattern of expression level changes may be indicative of a favorable response or an unfavorable one.

### DESCRIPTION OF THE DRAWINGS

FIG. 1 shows the IC50 values of cells treated with CDKI(7) or CDKI(8).

FIG. 2 shows differential gene expression in two different cell lines after treatment with CDKI(7).

FIG. 3 shows the reduction in expression of MEPCE when cells are treated with CDKI(8), CDKI(11) or Actinomycin D.

FIG. 4 shows differential expression of MEPCE and LARP7 protein levels after treatment with CDKI(7).

FIG. 5 shows differential gene expression of genes involved in the regulation of apoptosis in a lung cancer cell line treated with CDKI(7).

FIG. 6 shows the reduction of MEPCE expression in dog white blood cells when treated with CDKI(7).

FIG. 7 shows the reduction of MYC expression in dog white blood cells when treated with CDKI(7).

FIG. 8 shows the reduction of MCL1 expression in dog white blood cells when treated with CDKI(7).

FIG. 9 shows the reduction of MEPCE expression in dog white blood cells when treated with CDKI(12).

FIG. 10 shows no reduction in expression of MYC expression in dog white blood cells when treated with CDKI(12).

FIG. 11 shows no reduction of MCL1 expression in dog white blood cells when treated with CDKI(12).

### DESCRIPTION OF THE INVENTION

In one aspect, the disclosure relates to the analysis of a number of genes indentified in Table 2 that can act as pharmacodynamic (PD) markers. Increased or decreased expression of the PD marker after CDKI treatment indicates that the inhibitor is active. The response of the patient is evaluated based the detection of differential gene expression of at least one PD marker from Table 2. Detection and/or alteration in the level of expression of at least one PD marker compared to a baseline is indicative of the CDKI activity, and this can correlate with a response of the patient to the treatment. The pattern of expression level changes can be indicative of a favorable patient response or of an unfavorable one.

Accordingly, the disclosure provides for a method of monitoring the response of a patient to treatment with a Cyclin Dependent Kinase Inhibitor (CDKI), the method comprising:

a) administration of at least one CDKI;
b) measuring differential gene expression of at least one pharmacodynamic (PD) marker selected from Table 2 in a biological sample obtained from a patient who has been administered the CDKI; and
c) comparing the differential gene expression of the at least one PD marker with gene expression of the at least one PD marker in a control sample.

The method wherein the PD marker is selected from the group consisting of: MEPCE (SEQ ID NO: 1), MCL1 (SEQ ID NO: 3), MYC (SEQ ID NO: 5), HEXIM1 (SEQ ID NO: 7), LARP7 (SEQ ID NO: 9) or WHSC2 (SEQ ID NO: 11).

The method wherein the PD marker is MEPCE (SEQ ID NO:1).

The method wherein a nucleic acid or protein of at least one PD marker is measured.

The method wherein the gene expression of the at least one PD marker is reduced.

The method wherein the gene expression of at least two PD markers is measured.

The method further comprising obtaining a biological sample from the patient prior to the administration of the CDKI.

The method wherein the biological sample is obtained from lung cancer, melanoma, myeloma, breast cancer, glioblastoma, pancreatic cancer, thyroid cancer, ovarian cancer, bladder cancer, prostate cancer, liver cancer, colon cancer or PMBC.

The method wherein the CDKI inhibits CDK9.

The method wherein the CDKI is selected from Table 1.

The method wherein the CDKI was administered in a therapeutically effective amount.

The method wherein the therapeutically effective amount is adjusted for in subsequent administration of the CDKI to the patient.

The method wherein the differential expression of the PD marker is measured at least at two different time points.

The method wherein the steps b) and c) are repeated at 1 hour, 2 hours, 3 hours, 4 hours, 8 hours, 16 hours, 24 hours and 48 hours.

The method wherein two different CDKI are administered at step a).

The method wherein the two different CDKI are administered at the same time.

The method wherein the two different CDKI are administered at different time points.

A method of determining the sensitivity of a cell to a Cyclin Dependent Kinase Inhibitor (CDKI), the method comprising:

a) contacting a cell with at least one CDKI;
b) measuring differential gene expression of at least one pharmacodynamic (PD) marker selected from Table 2 in the cell contacted with the CDKI; and
c) comparing the differential gene expression with gene expression from an untreated or placebo treated control cell.

The method wherein the PD marker is selected from the group consisting of: MEPCE (SEQ ID NO: 1), MCL1 (SEQ ID NO: 3), MYC (SEQ ID NO: 5), HEXIM1 (SEQ ID NO: 7), LARP7 (SEQ ID NO: 9) or WHSC2 (SEQ ID NO: 11).

The method wherein the PD marker is MEPCE (SEQ ID NO:1).

The method wherein a nucleic acid or protein of at least one PD marker is measured.

The method wherein the gene expression of the at least one PD marker is reduced.

The method wherein the gene expression of at least two PD markers is measured.

The method wherein the cell is obtained from lung cancer, melanoma, myeloma, breast cancer, glioblastoma, pancreatic cancer, thyroid cancer, ovarian cancer, bladder cancer, prostate cancer, liver cancer, colon cancer or PMBC.

The method wherein the CDKI inhibits CDK9.

The method wherein the CDKI is selected from Table 1.

The method wherein the differential expression of the PD marker is measured at least at two different time points.

The method wherein the steps b) and c) are repeated at 1 hour, 2 hours, 3 hours, 4 hours, 8 hours, 16 hours, 24 hours and 48 hours.

The method wherein the cell is contacted by two different CDKI at step a).

The method wherein the cell is contacted by the two different CDKI at the same time.

The method wherein the cell is contacted by two different CDKI at different time points.

A method of screening for CDKI candidates the method comprising:

a) contacting a cell with a CDKI candidate;
b) measuring differential gene expression of at least one pharmacodynamic (PD) marker selected from Table 2 in the cell contacted with the CDKI candidate; and
c) comparing the differential gene expression of at least one PD marker of the cell contacted with the CDKI candidate with differential gene expression of at least one PD marker of a cell contacted with a CDKI taken from Table 1 and the differential gene expression of at least one PD marker of an untreated or placebo treated cell.

The method wherein the PD marker is selected from the group consisting of: MEPCE (SEQ ID NO: 1), MCL1 (SEQ ID NO: 3), MYC (SEQ ID NO: 5), HEXIM1 (SEQ ID NO: 7), LARP7 (SEQ ID NO: 9) or WHSC2 (SEQ ID NO: 11).

The method wherein the PD marker is MEPCE (SEQ ID NO:1).

The method wherein a nucleic acid or protein of at least one PD marker is measured.

The method wherein the gene expression of the at least one PD marker is reduced, indicating the CDKI candidate is a CDK inhibitor.

The method wherein the screening is for CDK9 inhibitors.

The method wherein the differential gene expression of the CDKI candidate is compared with the differential gene expression of a CDKI selected from Table 1.

The method wherein the cell is obtained from lung cancer, melanoma, myeloma, breast cancer, glioblastoma, pancreatic cancer, thyroid cancer, ovarian cancer, bladder cancer, prostate cancer, liver cancer, colon cancer or PMBC.

The method wherein the differential expression of the PD marker is measured at least at two different time points.

The method wherein the steps b) and c) are repeated at 1 hour, 2 hours, 3 hours, 4 hours, 8 hours, 16 hours, 24 hours and 48 hours.

The PD markers noted herein may be utilized to monitor patient response to treatment. For example, dosage amounts may be adjusted, additional therapies may be introduced, toxic response or other adverse events may be foreshadowed and forestalled, or treatment may be discontinued, depending upon the response of the patient to the CDKI as measured by the differential expression of PD markers disclosed in Table 2.

Definitions

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely exemplary and that equivalents of such are known in the art.

The terms “pharmacodynamic marker” or “PD marker” are used interchangeably herein. A pharmacodynamic marker is a gene and the presence, absence or differential expression of nucleic acid or polypeptide levels are used to determine if an administered CDKI is active. For example, after a cell is contacted with any CDKI, the mRNA expression of MEPCE gene is reduced when compared to MEPCE expression prior to contact with the CDKI inhibitor or a placebo treated/untreated control.

The terms “nucleic acid” and “polynucleotide” are used interchangeably and refer to a polymeric form of nucleotides of any length, either deoxyribonucleotides or ribonucleotides or analogs thereof. Polynucleotides can have any three-dimensional structure and may perform any function. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A “gene” refers to a polynucleotide containing at least one open reading frame (ORF) that is capable of encoding a particular polypeptide or protein after being transcribed and translated. A polynucleotide sequence may be used to identify larger fragments or full-length coding sequences of the gene with which they are associated. Methods of isolating larger fragment sequences are known to those of skill in the art.

A “gene product” or alternatively a “gene expression product” refers to the amino acids (e.g., peptide or polypeptide) generated when a gene is transcribed and translated.

The term “polypeptide” is used interchangeably with the term “protein” and in its broadest sense refers to a compound of two or more subunit amino acids, amino acid analogs, or peptidomimetics. The subunits may be linked by peptide bonds. In another embodiment, the subunit may be linked by other bonds, e.g., ester, ether, etc.

As used herein the term “amino acid” refers to either natural and/or unnatural or synthetic amino acids, and both the D and L optical isomers, amino acid analogs, and peptidomimetics. A peptide of three or more amino acids is commonly called an oligopeptide if the peptide chain is short. If the peptide chain is long, the peptide is commonly called a polypeptide or a protein.

The term “isolated” means separated from constituents, cellular and otherwise, in which the polynucleotide, peptide, polypeptide, protein, antibody or fragment(s) thereof, are normally associated within nature. In one aspect of this invention, an isolated polynucleotide is separated from the 3′ and 5′ contiguous nucleotides with which it is normally associated within its native or natural environment, e.g., on the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated,” “separated” or “diluted” polynucleotide, peptide, polypeptide, protein, antibody or fragment(s) thereof, is distinguishable from its naturally occurring counterpart in that the concentration or number of molecules per volume is greater in a “concentrated” version or less than in a “separated” version than that of its naturally occurring counterpart. A polynucleotide, peptide, polypeptide, protein, antibody, or fragment(s) thereof, which differs from the naturally occurring counterpart in its primary sequence or, for example, by its glycosylation pattern, need not be present in its isolated form since it is distinguishable from its naturally occurring counterpart by its primary sequence or, alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in PCR: A Practical Approach, M. MacPherson et al., IRL Press at Oxford University Press (1991). All processes of producing replicate copies of a polynucleotide, such as PCR or gene cloning, are collectively referred to herein as “replication.” A primer can also be used as a probe in hybridization reactions, such as Southern or Northern blot analyses (Sambrook et al., Molecular Cloning: A Laboratory Manual, 2nd edition (1989)).

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

“Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. However, as used herein, overexpression is an increase in gene expression and generally is at least 1.25 fold or, alternatively, at least 1.5 fold or, alternatively, at least 2 fold, or alternatively, at least 4 fold expression over that detected in a normal or control counterpart cell or tissue. As used herein, underexpression, is a reduction of gene expression and generally is at least 1.25 fold, or alternatively, at least 1.5 fold, or alternatively, at least 2 fold or alternatively, at least 4 fold expression under that detected in a normal or control counterpart cell or tissue. The term “differentially expressed” also refers to where expression in a cell or tissue is detected but expression in a control cell or tissue is undetectable.

A high expression level of the gene may occur because of over expression of the gene or an increase in gene copy number. The gene may also be translated into more protein because of deregulation or absence of a negative regulator.

A “gene expression profile” refers to a pattern of expression of at least one PD marker that recurs in multiple samples and reflects a property shared by those samples, such as tissue type, response to a particular treatment, or activation of a particular biological process or pathway in the cells. Furthermore, a gene expression profile differentiates between samples that share that common property and those that do not with better accuracy than would likely be achieved by assigning the samples to the two groups at random. A gene expression profile may be used to predict whether samples of unknown status share that common property or not. Some variation between the levels of at least one PD marker and the typical profile is to be expected, but the overall similarity of the expression levels to the typical profile is such that it is statistically unlikely that the similarity would be observed by chance in samples not sharing the common property that the expression profile reflects.

The term “cDNA” refers to complementary DNA, i.e. mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels, microarrays, and chips. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories), polyHIPE(R)™ resin (obtained from Aminotech, Canada), polyamide resin (obtained from Peninsula Laboratories), polystyrene resin grafted with polyethylene glycol (TentaGeIR™, Rapp Polymere, Tubingen, Germany), or polydimethylacrylamide resin (obtained from Milligen/Biosearch, California).

A polynucleotide also can be attached to a solid support for use in high throughput screening assays. PCT WO 97/10365, for example, discloses the construction of high density oligonucleotide chips. See also, U.S. Pat. Nos. 5,405,783; 5,412,087; and 5,445,934. Using this method, the probes are synthesized on a derivatized glass surface to form chip arrays. Photoprotected nucleoside phosphoramidites are coupled to the glass surface, selectively deprotected by photolysis through a photolithographic mask and reacted with a second protected nucleoside phosphoramidite. The coupling/deprotection process is repeated until the desired probe is complete.

As an example, transcriptional activity may be assessed by measuring levels of messenger RNA using a gene chip such as the Affymetrix HG-U133-Plus-2 GeneChips™. High-throughput, real-time quanititation of RNA of a large number of genes of interest thus becomes possible in a reproducible system.

The terms “stringent hybridization conditions” refers to conditions under which a nucleic acid probe will specifically hybridize to its target subsequence, and to no other sequences. The conditions determining the stringency of hybridization include: temperature, ionic strength, and the concentration of denaturing agents such as formamide. Varying one of these factors may influence another factor and one of skill in the art will appreciate changes in the conditions to maintain the desired level of stringency. An example of a highly stringent hybridization is: 0.015M sodium chloride, 0.0015M sodium citrate at 65-68 degrees C. or 0.015M sodium chloride, 0.0015M sodium citrate, and 50% formamide at 42 degrees C. (see Sambrook, supra). An example of a “moderately stringent” hybridization is the conditions of: 0.015M sodium chloride, 0.0015M sodium citrate at 50-65 degrees C. or 0.015M sodium chloride, 0.0015M sodium citrate, and 20% formamide at 37-50 degrees C. The moderately stringent conditions are used when a moderate amount of nucleic acid mismatch is desired. One of skill in the art will appreciate that washing is part of the hybridization conditions. For example, washing conditions can include 02.X-0.1X SSC/0.1% SDS and temperatures from 42-68 degrees C., wherein increasing temperature increases the stringency of the wash conditions.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Current Protocols in Molecular Biology, Ausubel et al., eds., (1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant.

The term “cell proliferative disorders” shall include dysregulation of normal physiological function characterized by abnormal cell growth and/or division or loss of function. Examples of “cell proliferative disorders” includes but is not limited to hyperplasia, neoplasia, metaplasia, and various autoimmune disorders, e.g., those characterized by the dysregulation of T cell apoptosis.

As used herein, the terms “neoplastic cells,” “neoplastic disease,” “neoplasia,” “tumor,” “tumor cells,” “cancer,” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures.

The term “cancer” refers to cancer diseases including, for example, carcinomas (e.g., lung cancer, melanoma, myeloid disorders (e.g., myeloid leukemia, multiple myeloma and erythroleukemia), lymphocytic leukemia, breast cancer, glioblastoma, pancreatic cancer, thyroid cancer, ovarian cancer, bladder cancer, prostate cancer, liver cancer, colon cancer, and sarcomas (e.g., osteosarcoma).

The term “PBMC” refers to peripheral blood mononuclear cells and includes “PBL”—peripheral blood lymphocytes.

“Suppressing” tumor growth indicates a reduction in tumor cell growth when compared to tumor growth without contact with CDKI compounds. Tumor cell growth can be assessed by any means known in the art, including, but not limited to, measuring tumor size, determining whether tumor cells are proliferating using a 3H-thymidine incorporation assay, measuring glucose uptake by FDG-PET (fluorodeoxyglucose positron emission tomography) imaging, or counting tumor cells. “Suppressing” tumor cell growth means any or all of the following states: slowing, delaying and stopping tumor growth, as well as tumor shrinkage.

A “composition” is a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives, for example; proteins, peptides, amino acids, lipids, and carbohydrates (e.g., sugars, including monosaccharides and oligosaccharides; derivatized sugars such as alditols, aldonic acids, esterified sugars and the like; and polysaccharides or sugar polymers), which can be present singly or in combination, comprising alone or in combination 1-99.99% by weight or volume. Carbohydrate excipients include, for example; monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

The term “carrier” further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-quadrature-cyclodextrin), polyethylene glycols, flavoring agents, antimicrobial agents, sweeteners, antioxidants, antistatic agents, surfactants (e.g., polysorbates such as TWEEN 20™ and TWEEN 80™), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Remington's Pharmaceutical Science., 15th Ed. (Mack Publ. Co., Easton (1975) and in the Physician's Desk Reference, 52nd ed., Medical Economics, Montvale, N.J. (1998).

An “effective amount” is an amount sufficient to effect beneficial or desired results. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, mice, simians, humans, farm animals, sport animals, and pets.

An “inhibitor” of cyclin D kinase as used herein diminishes the effect of the cyclin D kinases. This inhibition may include, for example, reduction of kinase activity or reduction of elongation of transcription.

A number of genes have now been identified as PD markers for CDKI. The decrease or increase of gene expression of one or more of the PD markers identified herein can be used to determine if the CDKI is having the desired effect, for example, the reduction or underexpression of gene expression after administration with a CDKI. As an example, after 2 hours of treatment with a CDKI, the reduction of gene expression of MEPCE will provide information whether the CDKI is active. A listing of CDKI PD markers is found in Table 2.

CDK inhibitors (CDKI) are compounds which are inhibitors of CDK9, and are useful in conjunction with the methods of the invention. Because CDK9 is a downstream effector of transcription elongation, CDKI are useful in pharmaceutical compositions for human or veterinary use where inhibition of CDK9 is indicated, e.g., in the treatment of tumors and/or cancerous cell growth. In particular, such compounds are useful in the treatment of human cancer, since the progression of these cancers is at least partially dependent upon transcription elongation and therefore is susceptible to treatment by the interruption of CDK9 activity. CDKI compounds are useful in treating, for example, carcinomas (e.g., lung cancer, melanoma, myeloid disorders (e.g., myeloid leukemia, multiple myeloma and erythroleukemia), lymphocytic leukemia, breast cancer, glioblastoma, pancreatic cancer, thyroid cancer, ovarian cancer, bladder cancer, prostate cancer, liver cancer, colon cancer, and sarcomas (e.g., osteosarcoma). A listing of CDKI compounds is found in Table 1.

TABLE 1
CDKI compounds

| | |
|---|---|
| 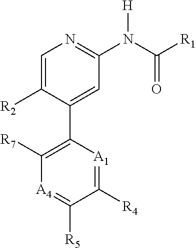 | CDKI(1) |
| 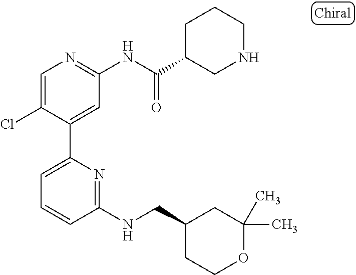 [SMILES: CC1(C)C[C@H](CNc2cccc(-c3cc(NC(=O)[C@@H]4CCCNC4)ncc3Cl)n2)CCO1] | CKDI(2) |
| 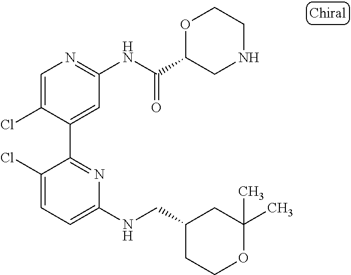 [SMILES: CC1(C)C[C@@H](CNc2ccc(Cl)c(-c3cc(NC(=O)[C@H]4CNCCO4)ncc3Cl)n2)CCO1] | CKDI(3) |
| 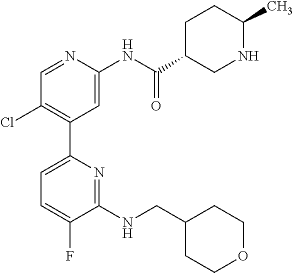 [SMILES: C[C@@H]1CC[C@@H](C(=O)Nc2cc(-c3ccc(F)c(NCC4CCOCC4)n3)c(Cl)cn2)CN1] | CKDI(4) |
| 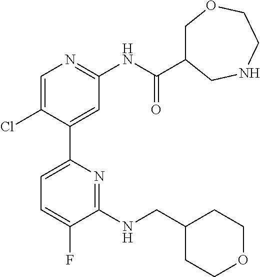 [SMILES: O=C(Nc1cc(-c2ccc(F)c(NCC3CCOCC3)n2)c(Cl)cn1)C1CNCCOC1] | CKDI(5) |
| 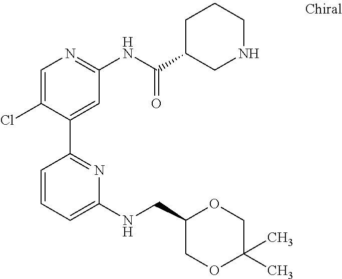 [SMILES: CC1(C)CO[C@H](CNc2cccc(-c3cc(NC(=O)[C@@H]4CCCNC4)ncc3Cl)n2)CO1] | CKDI(6) |
| 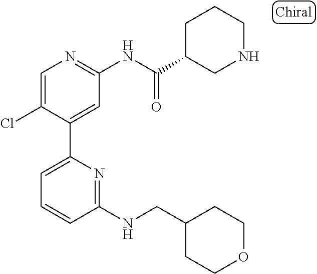 [SMILES: O=C(Nc1cc(-c2cccc(NCC3CCOCC3)n2)c(Cl)cn1)[C@@H]1CCCNC1] | CDKI(7) |
| 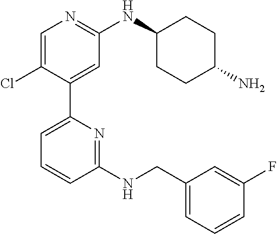 [SMILES: N[C@H]1CC[C@H](Nc2cc(-c3cccc(NCc4cccc(F)c4)n3)c(Cl)cn2)CC1] | CDKI(8) |
| 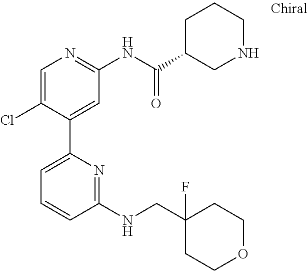 [SMILES: O=C(Nc1cc(-c2cccc(NCC3(F)CCOCC3)n2)c(Cl)cn1)[C@@H]1CCCNC1] | CKDI(9) |
| 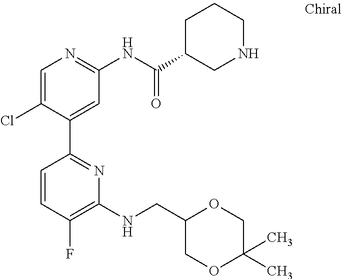 [SMILES: CC1(C)COC(CNc2nc(-c3cc(NC(=O)[C@@H]4CCCNC4)ncc3Cl)ccc2F)CO1] | CKDI(10) |
| 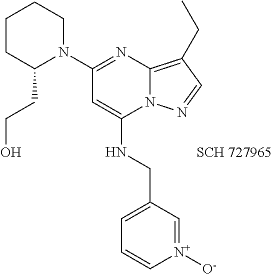 [SMILES: CCc1cnn2c(NCc3ccc[n+]([O-])c3)cc(N3CCCC[C@H]3CCO)nc12 ; "SCH 727965"] | CDKI(11) |
| 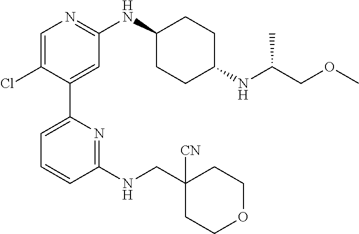 [SMILES: COC[C@@H](C)N[C@H]1CC[C@H](Nc2cc(-c3cccc(NCC4(C#N)CCOCC4)n3)c(Cl)cn2)CC1] | CDKI(12) |

Measurement of Gene Expression

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In one aspect, gene expression is detected and quantitated by hybridization to a probe that specifically hybridizes to the appropriate probe for that PD marker. The probes also can be attached to a solid support for use in high throughput screening assays using methods known in the art. WO 97/10365 and U.S. Pat. Nos. 5,405,783, 5,412,087 and 5,445,934, for example, disclose the construction of high density oligonucleotide chips which can contain one or more of the sequences disclosed herein. Using the methods disclosed in U.S. Pat. Nos. 5,405,783, 5,412,087 and 5,445,934, the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

In one aspect, the expression level of a gene is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device. See U.S. Pat. Nos. 5,578,832 and 5,631,734.

Alternatively any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

In one embodiment, the hybridized nucleic acids are detected by detecting one or more labels attached to the sample nucleic acids. The labels may be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 1251, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the coloured label.

The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

Detection of Polypeptides

Expression level of the PD marker can also be determined by examining the protein product. Determining the protein level involves (a) administration of at least one CDKI; and (b) measuring the amount of any immunospecific binding that occurs between an antibody that selectively recognizes and binds to the polypeptide of the PD marker in a sample obtained from a patient who has been administered the CDKI; and, (c) comparing to the amount of immunospecific binding of at least one PD marker with a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), western blot analysis, immunoprecipitation assays, immunofluorescent assays, flow cytometry, immunohistochemistry, confocal microscopy, enzymatic assays, surface plasmon resonance and PAGE-SDS.

Assaying for PD Markers and CDKI Treatment

Administration of a CDKI to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

PD markers are assayed for after CDKI administration in order to determine if the CDKI is active. In addition, PD markers can be assayed for in multiple timepoints after a single CDKI administration. For example, an initial bolus of CDKI is administered, and PD markers are assayed for at 1 hour, 2 hours, 3 hours, 4 hours, 8 hours, 16 hours, 24 hours and 48 hours after that first treatment.

PD markers can be assayed for after each CDKI administration, so if there are multiple CDKI administrations, then the PD markers can be assayed for after each administration. The patient could undergo multiple CDKI administrations and the PD markers then assayed at different timepoints. For example, a course of treatment may require administration of an initial dose of CDKI, a second dose a specified time period later, and still a third dose hours after the second dose. PD markers could be assayed for at 1 hour, 2 hours, 3 hours, 4 hours, 8 hours, 16 hours, 24 hours and 48 hours after administration of each dose of CDKI.

It is also within the scope of the disclosure that different PD markers are assayed for at different time points. Without being bound to any one theory, due to mechanism of action of the CDKI or of the PD marker, the response to the CDKI is delayed and the PD marker is assayed for at any time after administration. For example, a first PD marker is a marker for early responsiveness to the CDKI and is assayed for at any time within 0-4 hours of administration. A second PD marker could then be assayed for at any time within 4-8 hours after administration of the CDKI. An assay for at least one PD marker after each administration of CDKI will provide guidance as to the means, dosage and course of treatment.

Finally, there is administration of different CDKIs followed by assaying for at least one PD marker. In this embodiment, more than one CDKI is chosen from Table 1 and administered to the patient. At least one PD marker can then be assayed for after administration of each different CDKI. This assay can also be done at multiple timepoints after administration of the different CDKI. For example, a first CDKI could be administered to the patient and PD markers assayed at 1 hour, 2 hours, 3 hours, 4 hours, 8 hours, 16 hours, 24 hours and 48 hours. A second CDKI could then be administered and PD markers could be assayed for again at 1 hour, 2 hours, 3 hours, 4 hours, 8 hours, 16 hours, 24 hours and 48 hours.

Another aspect of the disclosure provides for a method of assessing for suitable dose levels of an CDKI, comprising monitoring the differential expression of at least one of the genes identified in Table 2 after administration of the CDKI. For example, after administration of a first bolus of CDKI, PD markers are analyzed and based on this result, an increase in CDKI dosage is recommended. After administration of the higher dosage of CDKI the analysis of the PD markers will determine whether the higher dose is providing the expected benefit, e.g., suppressing tumor growth.

Kits for assessing the activity of a CDKI can be made. For example, a kit comprising nucleic acid primers for PCR or for microarray hybridization for the genes listed in Table 2 can be used for assessing CDKI activity. Alternatively, a kit supplied with antibodies for at least one of the genes listed in Table 2 would be useful in assaying for CDKI activity or the resistance thereof.

It is well known in the art that cancers can become resistant to chemotherapeutic treatment, especially when that treatment is prolonged. Assaying for differential expression of PD markers can be done after prolonged treatment with any chemotherapeutic to determine if the cancer is sensitive to the CDKI. For example, kinase inhibitors such as Gleevec will strongly inhibit a specific kinase, but may also weakly inhibit other kinases. There are also other CDK inhibitors, different from the CDKIs described herein. If the patient has been previously treated with another kinase inhibitor or another type of CDK inhibitor, it is useful information to the patient to assay for the PD markers in Table 2 to determine if the tumor is sensitive to a CDKI. This assay can be especially beneficial to the patient if the cancer goes into remission and then re-grows or has metastasized to a different site.

Screening for CDK Inhibitors

It is possible to use the CDKI and PD markers of the invention to screen for other CDK inhibitors. This method comprises contacting a cell with a CDK inhibitor candidate, measuring the differential gene expression of at least one of the PD markers listed in Table 2 and comparing the result with the differential expression of a cell contacted with a CDKI of Table 1 as well as with the expression of at least one PD marker in a control cell. For example, the candidate CDK inhibitor will reduce PD marker expression at least as robustly as a CDKI listed in Table 1. The measurement of PD marker expression can be done by methods described previously, for example, PCR or microarray analysis.

TABLE 2

| | | SEQ ID NO. |
|---|---|---|
| Gene Name | Accession number | (nucleotide/protein) |
| MEPCE | NM_181708 | SEQ ID NO. 1/SEQ ID NO. 2 |
| MCL1 | NM_021960 | SEQ ID NO. 3/SEQ ID NO. 4 |
| MYC | NM_002467 | SEQ ID NO. 5/SEQ ID NO. 6 |
| HEXIM | NM_006460 | SEQ ID NO. 7/SEQ ID NO. 8 |
| LARP7 | NM_016648 | SEQ ID NO. 9/SEQ ID NO. 10 |
| WHSC2 | NM_005663 | SEQ ID NO. 11/SEQ ID NO. 12 |

### EXAMPLES

### Example 1

Five cell lines were analyzed: NCI-H929, a multiple myeloma cell line (ATCC Cat. # CRL-9068); NCI-H441, a lung papillary adenocarcinoma cell line (ATCC Cat. # HTB-174); A375, a melanoma cell line (ATCC Cat. # CRL-1619); A2058, a melanoma cell line (ATCC Cat. #CRL-1147) and U-87-MG, a glioblastoma cell line (ATCC Cat. #HTB-14). Cell lines were grown in the medium recommended by ATCC and treated as follows:

NCI-H929: 2 hours: DMSO, 200 nM CKDI(8) or 500 nM CKDI(8).

NCI-H441 and A375: 0 timepoint: Untreated, harvested when compound is added to the other plates.

2 hours: DMSO, 200 nM CKDI(8) or 500 nM CKDI(8) or 500 nM CKDI(7) (3 plates each, total 12 plates).

8 hours: DMSO, 200 nM CKDI(8) or 500 nM CKDI (8) or 500 nM CKDI(7) (3 plates each, total 12 plates).

16 hours: DMSO, 200 nM CKDI(8) or 500 nM CKDI(8) or 500 nM CKDI(7) (3 plates each, total 12 plates).

A2058, U-87-MG

0 timepoint: Untreated, harvested when compound is added to the other plates (3 plates).

2 hours: DMSO, 500 nM CKDI(8) (3 plates each, total 6 plates).

8 hours: DMSO, 500 nM CKDI(8) (3 plates each, total 6 plates).

16 hours: DMSO, 500 nM CKDI(8) (3 plates each, total 6 plates).

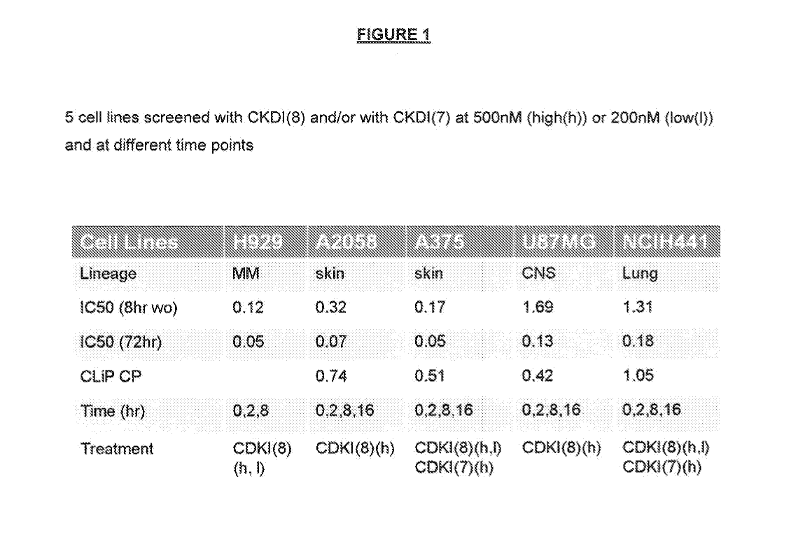

The IC50 result of this analysis is shown in FIG. 1. RNA was prepared for microarray profiling using the Affymetrix GeneChip ArrayStation ™ (Affymetrix, Santa Clara, Calif. USA) running the manufacturer's recommended protocols. The target products were hybridized to Affymetrix ™ U133-Plus-2 whole genome microarrays and scanned with an Affymetrix GeneChip Scanner 3000™. Bioinformatics analysis was performed on the raw data to provide the results. The manufacturer's recommended quality control criteria were examined during the laboratory processing and data analysis to ensure high-quality results.

An average expression level was determined for each probeset at each time-point and treatment condition by computing the geomean expression level across replicate tumors. For purposes of this experiment, probesets were identified as significantly differential if the ratio of CDKI treated/vehicle-only control was greater than 2-fold up or down with a p-value less than 0.001 in at least one time point. The p-value was determined by a two-tailed t-test assuming unequal variances (Satterwaithe's approximation). The probesets were mapped to genes by blasting the target probe sequences, supplied by Affymetrix, against GenBank™.

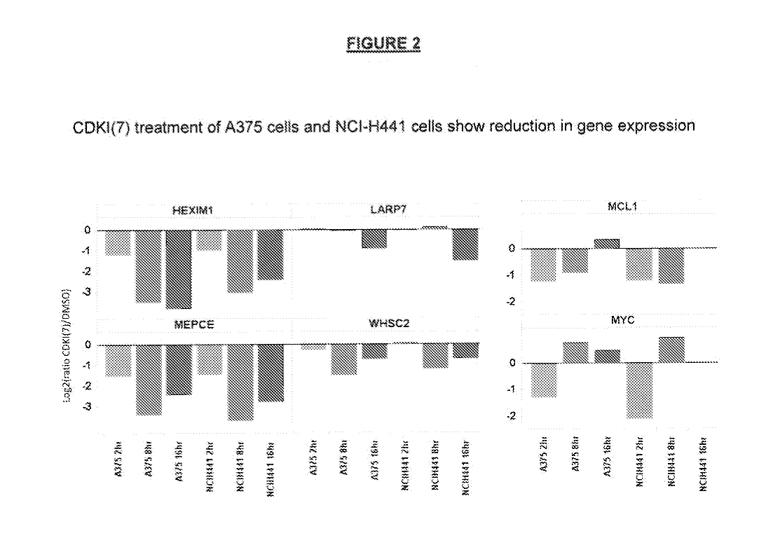

Of the genes probed by microarray, the expression of the PD markers MEPCE, MCL, MYC, HEXIM, LARP7 and WHSC2 are reduced indicating that that the CDKI is active. This is shown in FIG. 2, where A375 cells that are sensitive to CDKI were treated with CDKI(7) and the reduction of gene expression was determined at 2, 8 and 16 hours of treatment. As shown in FIG. 2, the expression of certain genes, for example MEPCE, has the greatest amount of reduction at the 8 hour time point.

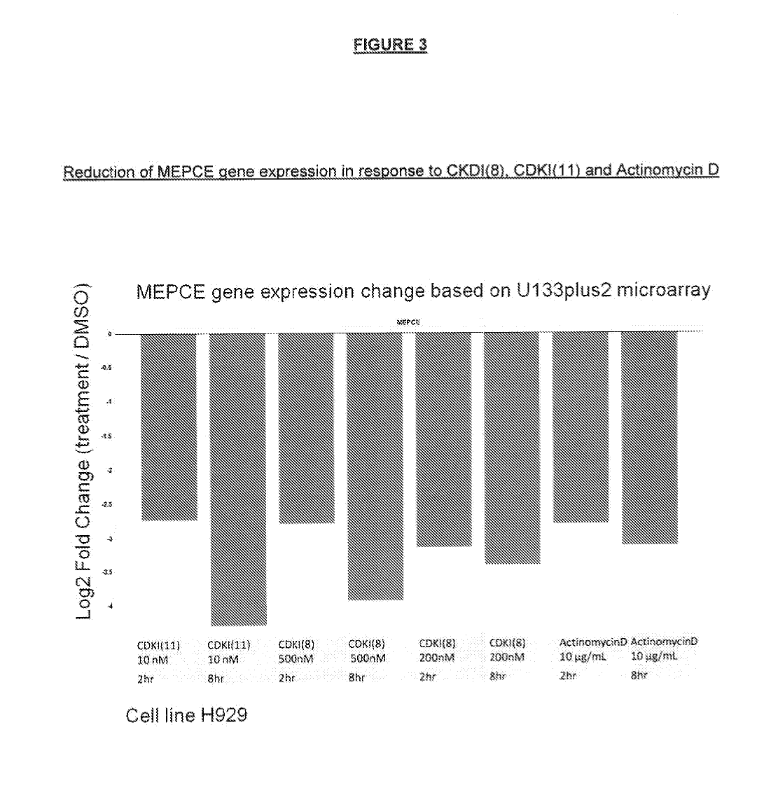

FIG. 3 shows that MEPCE expression is reduced upon treatment with CDKI(8) which is a specific CDK9 inhibitor, and this reduction in expression was compared with CDKI(11) which is a pan-CDK inhibitor that strongly inhibits all CDKs. CDKI(11) is also known as SCH727965 or Dinaciclib® (Mol. Cancer Ther. 2010, 9(8):2344-2353). As shown in FIG. 3, both CDKI(8) and CDKI(11) reduce the expression of MEPCE indicating that MEPCE is a PD marker for any CDK9 inhibitor. Actinomycin D, which also reduces transcription elongation, was also used, and FIG. 3 shows that treatment with this compound also reduces the expression of MEPCE.

### Example 2

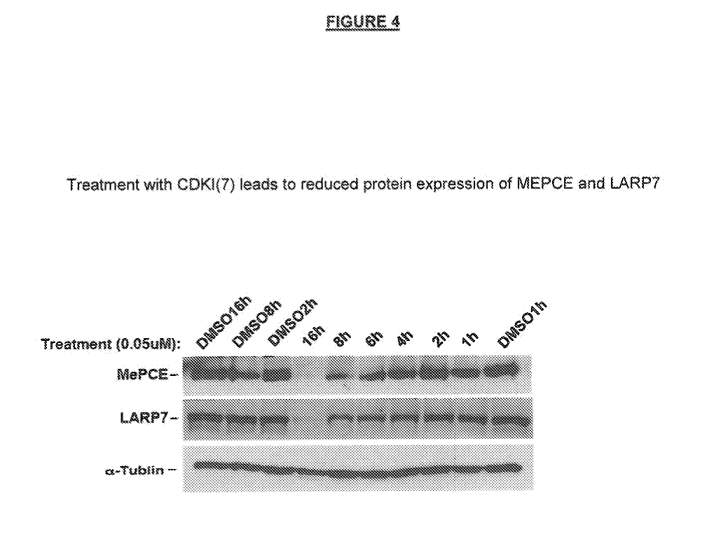

After treatment with a CDKI, cells were lysed in modified RIPA buffer containing 50 mM TRIS pH 7.4, 150 mM NaCl, 1 mM EDTA, 1% Brij-35, 0.1% deoxycholate, protease inhibitors (Roche Molecular Biochemicals, Mannheim, Germany). SDS-PAGE (4-12% gel) was used to resolve the proteins in the lysate. After electrophoresis, the proteins were electrotransferred onto a polyvinylidene fluoride microporous membrane and immunodetected using standard procedures. FIG. 4 is a Western blot of the multiple myeloma line NCI-H929, treated with CDKI(7) or DMSO and gene expression analyzed at certain time points during the treatment. As shown in the blot, gene expression of MEPCE is reduced over time, with the 16 hour time point showing very little to undetectable levels of protein expression. Thus, the reduction in expression of genes in response to CDKI seen at the mRNA level by microarray is confirmed at the protein level by Western blotting.

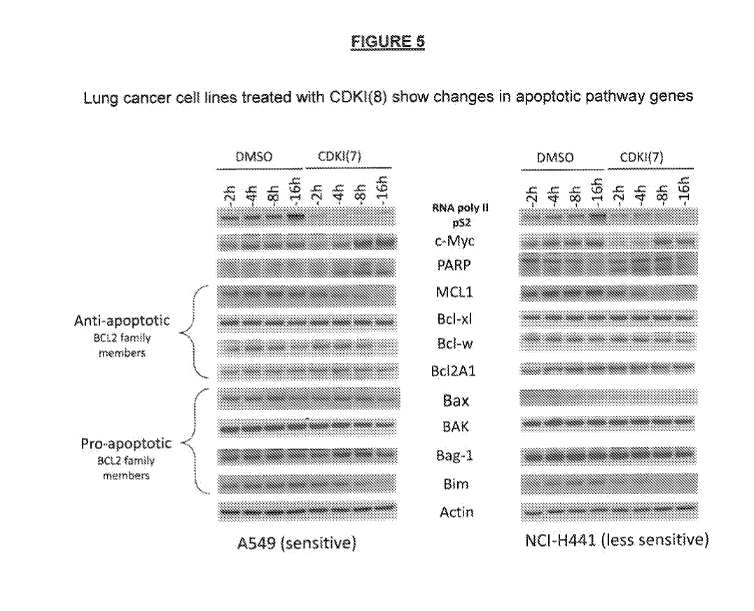

FIG. 5 is a Western blot comparing the reduction of expression of anti-apoptotic and pro-apoptotic family members after treatment with CDKI(8). For example, the expression of MCL1 protein is reduced 2 hours after treatment and is further reduced after 16 hour exposure to CDKI(8).

### Example 3

As part of a dog toxicology study, CDKI(7) was administered to dogs. White blood cells were harvested and MEPCE, MYC and MCL1 gene expression was assessed as PD markers. mRNA levels of each of these PD markers were measured by TaqMan® at various time points after CDKI(7) treatment.

The study had three groups of three dogs each. Dogs in Group 1 (#101, #102, and #103) were treated with CDKI(7) at 0.5 mg/kg, dogs in Group 2 (#104, #105, and #106) were treated at 1 mg/kg CDKI(7) and dogs in Group 3 (#107, #108, and #109) were treated at 2 mg/kg CDKI(7). Samples were collected before treatment (0 hour), 3 hours, 7 hours and 24 hours after treatment for a total of 12 samples per group. Blood samples were received frozen in QIAGEN RNA protect® Animal Blood tubes (Cat. # 76554 QIAGEN-Valencia, Calif., USA). RNA samples were extracted using the QIAGEN RNeasy® Protect Animal Blood kit (Cat. # 73224). RNA samples were quantified using the RNA Quant-iT RNA® assay from Invitrogen (Cat. # Q32852 Invitrogen—Grand Island, N.Y., USA) and read on an Invitrogen Qubit fluorometer® (Cat. # Q32857). First strand cDNA synthesis was performed on 500 ng of RNA in a reaction volume of 20 μl using a High Capacity cDNA Reverse Transcription Kit® from Applied Biosystems (Cat. # 4368814 Applied Biosystems, Carlsbad, Calif., USA). The cDNA samples were then diluted to obtain a 10 ng cDNA input for the TaqMan® reaction. TaqMan® reactions were run in triplicates using a TaqMan® Gene Expression Master Mix from Applied Biosystems (Cat. # 4369016) on an ABI 7500® machine. The probe sets used to assay for MEPCE, MYC and MCL1 expression are shown in Table 3. The 18S rRNA level was used as an endogenous control for variations in cDNA input. The relative quantification method was used to analyze the data; this method uses one sample as a calibrator sample and express mRNA levels relative to that sample. The results were expressed as RQ values (Relative Quantification) with RQ Min and RQ Max values determining a 95% confidence interval for the real RQ value. The samples were grouped according to the dose and each group was treated as a separate experiment with one of the pre-dose samples used as a calibrator sample. The logarithm of the RQ values are plotted so that increases or decreases from the initial value can be more easily represented; on a logarithmic scale: a 10-fold increase or 10-fold decrease in RQ value are represented as +1 and −1 respectively. Note that the last data point for dog #108 was removed due to greatly reduced yield of RNA.

TABLE 3
TaqMan ® gene expression assays used

| Gene symbol | Applied Biosystems Assay ID |
|---|---|
| MEPCE | Cf02683604_m1 |
| MYC | Cf02628821_m1 |
| MCL1-1 | Cf02713468_m1 |
| MCL1-2 | Cf02622286_m1 |
| 18S rRNA | 4319413E |

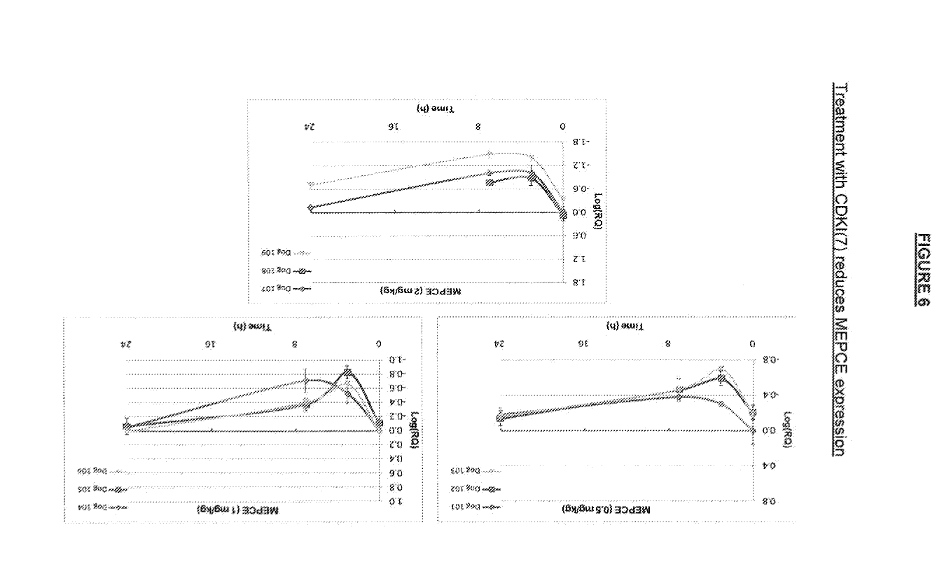

Previous Examples show that the MEPCE mRNA is particularly sensitive to CDK9 inhibition by any CDKI (see FIG. 2). In addition, MEPCE is part of the 7SK snRNA complex which sequesters P-TEFb, the positive transcription elongation factor that binds CDK9, to keep it in an inactive form. The mRNA levels were determined by TaqMan® using the relative quantification method. The calibrator samples are dog #101 at 0 hour timepoint for the 0.5 mg/kg dose of CDKI(7), dog #104 at 0 hour timepoint for the 1 mg/kg dose and dog #107 at 0 hour timepoint for the CDKI(7) 2 mg/kg dose. The logarithm (base 10) of the RQ values is plotted with the error bars representing the 95% confidence interval. Results for the MEPCE mRNA are shown in FIG. 6. There is clear down regulation in MEPCE at all three doses of CDKI. The amplitude and the duration of the down regulation are dose-dependent; at 0.5 mg/kg CDKI(7) the maximum down regulation is 3.2-fold at 3 hours, at 1 mg/kg CDKI(7) it is 5.2-fold at 7 hours and at 2 mg/kg it is 14-fold at 7 hours. At 24 hours, MEPCE mRNA levels are back to pre-treatment levels, except at the 2 mg/kg dose, where one of the dogs (#109) still displays some degree of down regulation.

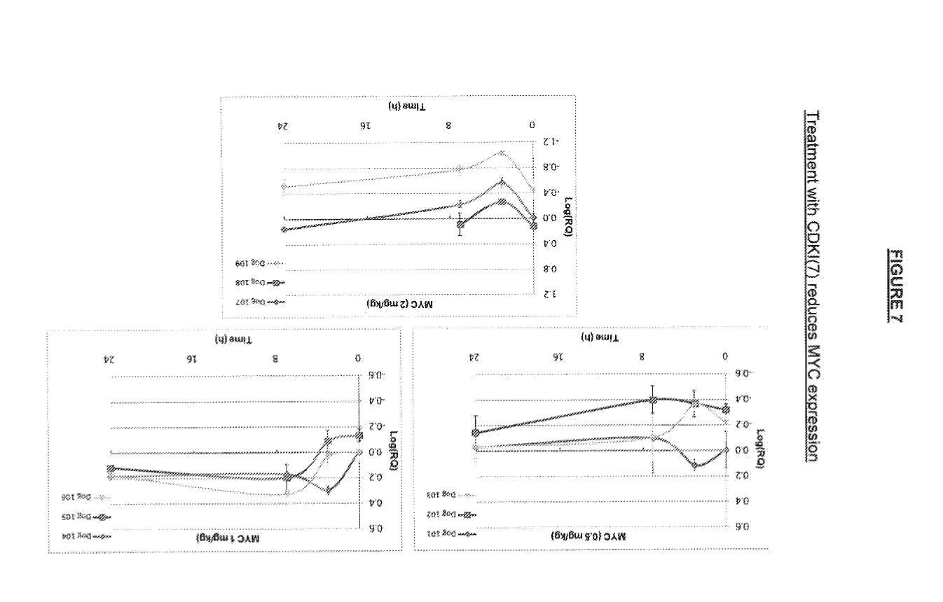

The MYC transcription factor is a known oncogene and previous Examples have shown that its mRNA is short-lived and sensitive to CDK9 inhibition (see FIG. 2). The MYC mRNA levels were determined by TaqMan® using the relative quantification method. The calibrator samples are dog #101 at 0 hour timepoint for the 0.5 mg/kg dose of CDKI(7), dog #104 at 0 hour timepoint for the 1 mg/kg CDKI(7) dose and dog #107 at 0 hour timepoint for the 2 mg/kg CDKI(7) dose. The logarithm (base 10) of the RQ values is plotted with the error bars representing the 95% confidence interval. The MYC mRNA (see FIG. 7) shows a consistent down regulation only at the 2 mg/kg dose. MYC expression from all three dogs show down regulation at 3 hours (maximum 3.8-fold) and 2 out of three at 7 hours. At lower doses there is either no significant down regulation (0.5 mg/kg) or a small increase (2-fold) at 1 mg/kg for the 3 or 7 hour time points. In all cases mRNA levels trended back to pre-treatment levels by 24 hours.

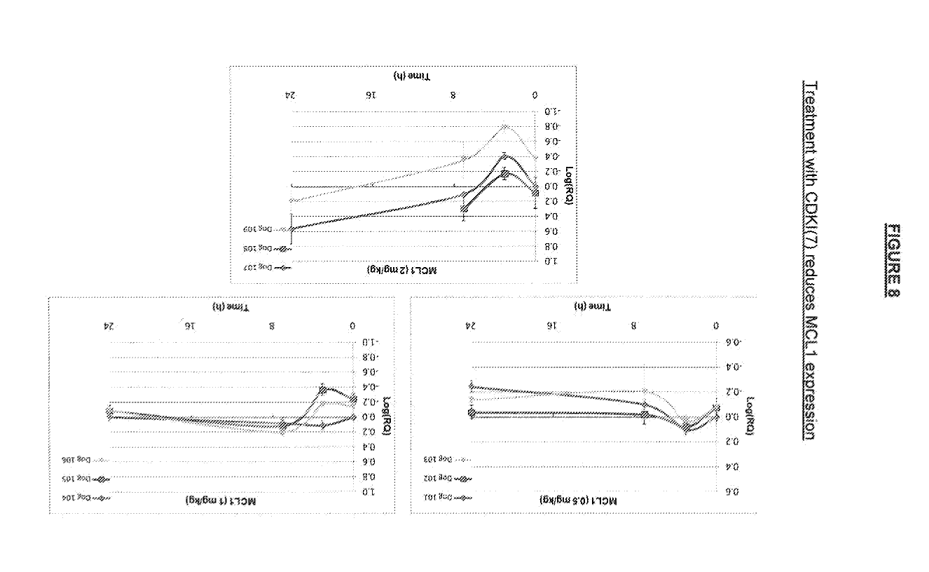

The MCL1 gene encodes a pro-survival factor that is often amplified in cancer and we have previously shown that its mRNA is short-lived and sensitive to CDK9 inhibition (see FIG. 2). The mRNA levels were determined by TaqMan® using the relative quantification method. The calibrator samples are dog #101 at 0 hour timepoint for the 0.5 mg/kg dose of CDKI(7), dog #104 at 0 hour timepoint for the 1 mg/kg dose and dog #107 at 0 hour timepoint for the 2 mg/kg dose. The logarithm (base 10) of the RQ values is plotted with the error bars representing the 95% confidence interval. The MCL1 mRNA (see FIG. 8) shows a consistent down regulation only at the 2 mg/kg dose. MCL1 expression in all three dogs show down regulation at 3 hours (maximum 2.7-fold) but not at 7 hours. At lower doses there is either no significant down regulation (0.5 mg/kg) or a small increase (2-fold) at 1 mg/kg for the 7 hour time point in two out of three dogs.

Three mRNAs were measured as PD markers for the effect of CDKI(7). The MEPCE mRNA was the most sensitive of the three markers, showing clear down regulation measurable at all three doses. The amplitude and the duration of the response were both dose proportional, reaching a maximum down regulation of 14-fold at 7 hours. Based on the experiments in cell lines as discussed above, it is clear that MEPCE is a PD marker for any molecule or compound that inhibits CDK9. MEPCE down regulation was shown in this study with CDKI(7), and the experiments above show reduction in MEPCE reduction with CDKI(8), CDKI(11) and CDKI(12), as shown below. The MYC mRNA was the second most sensitive marker; it was not significantly down regulated at 0.5 or 1 mg/kg, but 2 mg/kg dose of

CDKI(7) did reduce the expression of MYC. The down regulation was stronger and more consistent at 3 hours than at 7 hours. The amplitude of the down regulation was smaller than for MEPCE, with a maximum of 3.8-fold versus 14-fold for MEPCE. Finally, the MCL1 mRNA was the least sensitive of the three markers. It was down regulated only at 2 mg/kg, but only at the 3 hour time point and with less amplitude than for the MYC mRNA (maximum 2.7-fold versus 3.8-fold).

This experiment also indicates that if the CDKI is being administered to a patient as a therapeutic, that non-cancerous tissue can be assayed in order to determine if the CDKI is having an effect. For example, if a patient with melanoma is administered a CDKI for the purposes of reducing the melanoma, white blood cells from the patient's peripheral blood (PBMCs) can be assayed for MEPCE, as was done in this dog study. This means that tissue biopsies of cancerous and normal tissues are not strictly necessary to determine if the CDKI is active. A series of blood draws and TaqMan® assays can provide this information.

### Example 4

As part of a dog toxicology study, the pharmacodynamic markers MEPCE, MYC and MCL1 were measured by TaqMan® in white blood cells at various time points after CDKI(12) treatment. These mRNAs have previously been shown to be modulated by CDK9 inhibitors and CDKI in mice and rats, both in xenografts and in white blood cells.

The study had three groups of three dogs each. Dogs in Group 1 (#1001, #1002, and #1003) were treated with vehicle, dogs in Group 3 (#3001, #3002, and #3003) were treated with CDKI(12) at 0.1 mg/kg and dogs in Group 5 (#5001, #5002, and #5003) were treated with CDKI(12) at 0.15 mg/kg. Samples were collected before treatment 0 hour timepoint; 4 hours, 8 hours and 24 hours after treatment, for a total of 12 samples per group. Blood samples were received frozen in QIAGEN RNAprotect® Animal Blood tubes (Cat. #76554). RNA samples were extracted using the QIAGEN RNeasy® Protect Animal Blood kit (Cat. #73224). RNA samples were quantified using the RNA Quant-iT RNA assay® from Invitrogen (Cat. #Q32852) and read on an Invitrogen Qubit fluorometer (Cat. #Q32857). First strand cDNA synthesis was performed on 500 ng of RNA in a reaction volume of 20 μl using a High Capacity cDNA Reverse Transcription Kit from Applied Biosystems (Cat. #4368814). The cDNA samples were then diluted to 2 ng/μl (10 ng in 5 μl in each reaction). Each TaqMan® reaction contained: 5 μl of cDNA, 1.25 μl of each probe (18S rRNA and gene of interest), 5 μl of water and 12.5 μl of TaqMan® Gene Expression Master Mix from Applied Biosystems (Cat. #4369016) for a total volume of 25 μl. TaqMan® reactions were run in triplicates on an ABI 7500® machine. The probes used are shown in Table 3 (see Example 3 above). The 18S rRNA level was used as an endogenous control to correct for variations in cDNA input. The relative quantification method was used to analyze the data; this method uses one sample as a calibrator sample and express mRNA levels relative to that sample. The results are expressed as RQ values (Relative Quantification) with RQ Min and RQ Max values determining a 95% confidence interval for the RQ value. The samples were grouped according to treatment and time point and data from three dogs, each measured in triplicate, were averaged. The vehicle group at 0 hour timepoint was used as the calibrator sample thus allowing us to measure changes in mRNA levels in response to the test article at various time points. The logarithm of the RQ values are plotted so that increases or decreases from the initial value can be more easily represented; on a logarithmic scale a 10-fold increase or 10-fold decrease in RQ value are represented as +1 and −1 respectively.

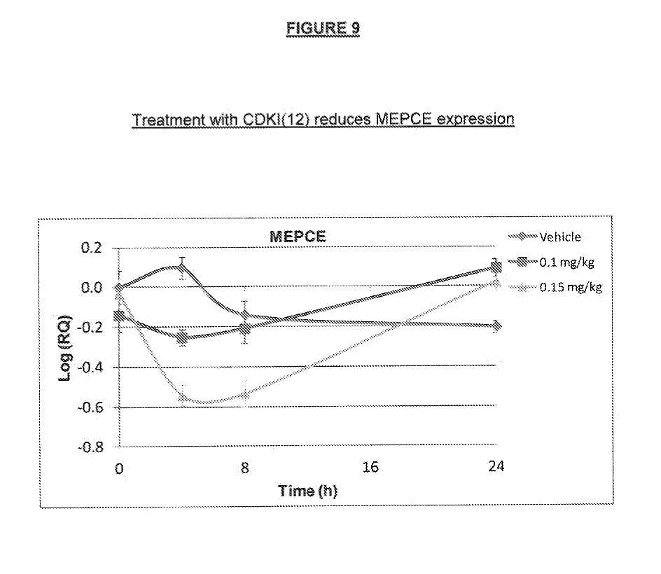

We have previously shown that the MEPCE mRNA is particularly sensitive to CDK9 inhibition and any CDKI (see Examples above). In addition, MEPCE is part of the 7SK snRNA complex which sequesters P-TEFb, the positive transcription elongation factor binds CDK9, to keep it in an inactive form. The mRNA levels were determined by TaqMan® using the relative quantification method. The calibrator sample is vehicle at 0 hour timepoint. The logarithm (base 10) of the RQ values is plotted with the error bars representing the 95% confidence interval. Results for the MEPCE mRNA are shown in FIG. 9. There is clear down regulation of MEPCE at the highest dose of CDKI(12) (0.15 mg/kg). The mRNA level is reduced at 4 and 8 hours and returns to pretreatment levels by 24 hours. There is less down regulation at 0.1 mg/kg of CDKI(12) but the curve parallels that of the 0.15 mg/kg group.

The MYC transcription factor is a known oncogene and we have previously shown that its mRNA is short-lived and sensitive to CDK9 inhibition. The mRNA levels were determined by TaqMan® using the relative quantification method. The calibrator sample is vehicle at time 0. The logarithm (base 10) of the RQ values is plotted with the error bars representing the 95% confidence interval.

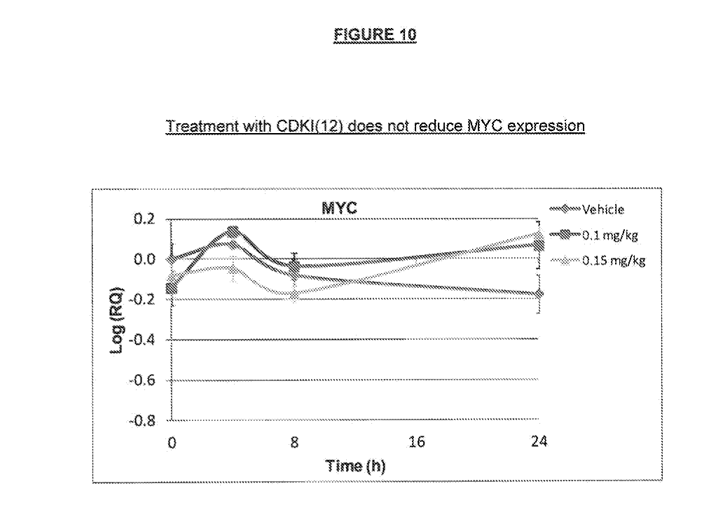

The MYC mRNA (see FIG. 10) does not show any significant down regulation at any of the tested doses.

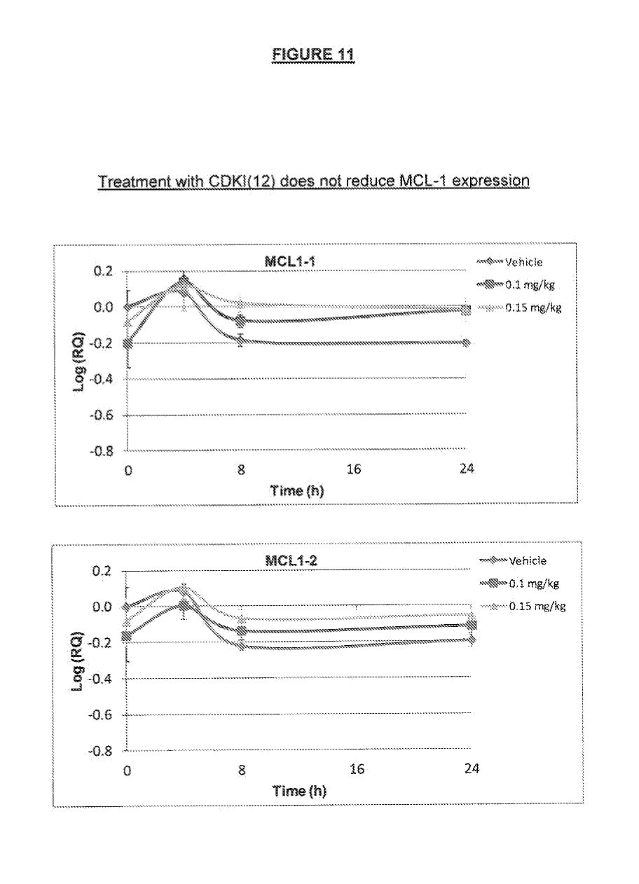

The MCL1 gene encodes a pro-survival factor that is often amplified in cancer and we have previously shown that its mRNA is short-lived and sensitive to CDK9 inhibition and CDKI. Two probes were used for the MCL1 mRNA (see Table 3 above), probe Cf02713468_m1 (MCL1-1) and probe Cf02622286_m1 (MCL1-2). Both probes show very similar results. The mRNA levels were determined by TaqMan® using the relative quantification method. The calibrator sample is vehicle at 0 hour timepoint. The logarithm (base 10) of the RQ values is plotted with the error bars representing the 95% confidence interval. The MCL1 mRNA (see FIG. 11) does not show any significant down regulation at any of the tested doses.

In summary, three different PD markers were assayed after the administration of CDKI(12). MEPCE was the most sensitive of the three markers; showing clear down regulation at 0.15 mg/kg and at 0.1 mg/kg of CDKI(12) respectively. The mRNA levels were reduced at 4 and 8 hours and returned to pretreatment levels by 24 hours. The MYC and MCL1 mRNAs were not significantly down regulated at the tested doses. As shown in Example 3 with CDKI(7), MYC and MCL1 require higher doses to be modulated, and this may be the case with CDKI(12).

1. A method of monitoring the response of a patient to treatment with a Cyclin Dependent Kinase Inhibitor (CDKI), the method comprising:
a) administration of at least one CDKI;
b) measuring differential gene expression of at least one pharmacodynamic (PD) marker selected from Table 2 in a biological sample obtained from a patient who has been administered the CDKI; and
c) comparing the differential gene expression of the at least one PD marker with gene expression of the at least one PD marker in a control sample.

2. The method of claim 1, wherein the PD marker is selected from the group consisting of: MEPCE (SEQ ID NO: 1), MCL1 (SEQ ID NO: 3), MYC (SEQ ID NO: 5), HEXIM1 (SEQ ID NO: 7), LARP7 (SEQ ID NO: 9) or WHSC2 (SEQ ID NO: 11).

3. The method of claim 1, wherein the PD marker is MEPCE (SEQ ID NO:1).

4. The method of claim 1, wherein a nucleic acid or protein of at least one PD marker is measured.

5. The method of claim 1, wherein the gene expression of the at least one PD marker is reduced.

6. The method of claim 1, wherein the gene expression of at least two PD markers is measured.

7. The method of claim 1 further comprising obtaining a biological sample from the patient prior to the administration of the CDKI.

8. The method of claim 1, wherein the biological sample is obtained from lung cancer, melanoma, myeloma, breast cancer, glioblastoma, pancreatic cancer, thyroid cancer, ovarian cancer, bladder cancer, prostate cancer, liver cancer, colon cancer or PMBC.

9. The method of claim 1, wherein the CDKI inhibits CDK9.

10. The method of claim 1, wherein the CDKI is selected from Table 1.

11. The method of claim 1, wherein the CDKI was administered in a therapeutically effective amount.

12. The method of claim 10, wherein the therapeutically effective amount is adjusted for in subsequent administration of the CDKI to the patient.

13. The method of claim 1, wherein the differential expression of the PD marker is measured at least at two different time points.

14. The method of claim 1, wherein the steps b) and c) are repeated at 1 hour, 2 hours, 3 hours, 4 hours, 8 hours, 16 hours, 24 hours and 48 hours.

15. The method of claim 1, wherein two different CDKI are administered at step a).

16. The method of claim 14, wherein the two different CDKI are administered at the same time.

17. The method of claim 14, wherein the two different CDKI are administered at different time points.

18. A method of determining the sensitivity of a cell to a Cyclin Dependent Kinase Inhibitor (CDKI), the method comprising:
a) contacting a cell with at least one CDKI;
b) measuring differential gene expression of at least one pharmacodynamic (PD) marker selected from Table 2 in the cell contacted with the CDKI; and
c) comparing the differential gene expression with gene expression from an untreated or placebo treated control cell.

19. The method of claim 18, wherein the PD marker is selected from the group consisting of: MEPCE (SEQ ID NO: 1), MCL1 (SEQ ID NO: 3), MYC (SEQ ID NO: 5), HEXIM1 (SEQ ID NO: 7), LARP7 (SEQ ID NO: 9) or WHSC2 (SEQ ID NO: 11).

20. The method of claim 18, wherein the PD marker is MEPCE (SEQ ID NO:1).

21. The method of claim 18, wherein a nucleic acid or protein of at least one PD marker is measured.

22. The method of claim 18, wherein the gene expression of the at least one PD marker is reduced.

23. The method of claim 18, wherein the gene expression of at least two PD markers is measured.

24. The method of claim 18, wherein the cell is obtained from lung cancer, melanoma, myeloma, breast cancer, glioblastoma, pancreatic cancer, thyroid cancer, ovarian cancer, bladder cancer, prostate cancer, liver cancer, colon cancer or PMBC.

25. The method of claim 18, wherein the CDKI is selected from Table 1.

26. The method of claim 18, wherein the differential expression of the PD marker is measured at least at two different time points.

27. The method of claim 18, wherein the steps b) and c) are repeated at 1 hour, 2 hours, 3 hours, 4 hours, 8 hours, 16 hours, 24 hours and 48 hours.

28. The method of claim 18, wherein the cell is contacted by two different CDKI at step a).

29. The method of claim 18, wherein the cell is contacted by the two different CDKI at the same time.

30. The method of claim 18, wherein the cell is contacted by two different CDKI at different time points.

31. -40. (canceled)

41. A kit used in a method of monitoring the response of a patient to treatment with a CDKI, the method comprising:
a) administration of at least one CDKI;
b) measuring differential gene expression of at least one pharmacodynamic (PD) marker selected from Table 2 in a biological sample obtained from a patient who has been administered the CDKI;
c) comparing the differential gene expression of the at least one PD marker with gene expression of the at least one PD marker in a control sample; and
wherein the kit comprises reagents for carrying out step b).
